(19) United States
(12) Patent Application Publication
(10) Pub. No.: US 2017/0106210 A1
(43) Pub. Date: Apr. 20, 2017

(54) METHOD AND MAGNETIC RESONANCE APPARATUS FOR PLANNING RADIOTHERAPY FOR A PATIENT

(71) Applicant: Siemens Healthcare GmbH, Erlangen (DE)

(72) Inventors: David Grodzki, Erlangen (DE); Arne Hengerer, Moehrendorf (DE); Michael Kaus, Nuernberg (DE); Sebastian Schmidt, Weisendorf (DE)

(73) Assignee: Siemens Healthcare GmbH, Erlangen (DE)

(21) Appl. No.: 15/292,217

(22) Filed: Oct. 13, 2016

(30) Foreign Application Priority Data
Oct. 15, 2015 (DE) 102015220077.9

(51) Int. Cl. A61N 5/10; G01R 33/483; A61B 5/055; G01R 33/48

(52) U.S. Cl. CPC A61N 5/1039; G01R 33/4808; G01R 33/483; A61B 5/055

(57) ABSTRACT

In a method and magnetic resonance apparatus for planning radiotherapy for a patient, quantitative magnetic resonance measurement data of a planning volume in the patient are acquired using a quantitative magnetic resonance method, a three-dimensional distribution of values of an electron density parameter in the planning volume are determined in a processor based on the acquired quantitative magnetic resonance measurement data, and a radiotherapy plan is calculated using the three-dimensional distribution of the values of the electron density parameter.

### BACKGROUND OF THE INVENTION

Field of the Invention

The invention concerns a method for planning radiotherapy for a patient, and a radiotherapy planning computer, a magnetic resonance apparatus and a computer program product.

Description of the Prior Art

During beam therapy, target tissue, such as a tumor, of a patient is irradiated using ionizing radiation. External beam therapy, which is the irradiation of the body of the patient from outside the body, is known for this purpose. Internal beam therapy, also referred to as brachytherapy, is also known. During brachytherapy beam sources, which include radioactive substances, are introduced into the body of the patient in order to damage or destroy the target tissue locally in the body of the patient.

It is known that beam therapy for a patient can be planned and/or monitored by means of imaging. To this end a radiotherapy plan is generally produced with the use of medical image data acquired from the patient using a three-dimensional imaging method. Image data acquired using computed tomography (CT image data) are generally used for this purpose. The CT image data can be used to determine the target volume for radiotherapy and as well as to locate surrounding tissue that has to be preserved, such as neural tissue. The intensity values of the image voxels of the image data (quantified in units known as Hounsfield units) also represent a close approximation of electron density at the corresponding site in the body of the patient, because the intensity values of the image voxels are based on absorption of the x-ray radiation at the associated sites. This allows the CT image data to be converted to an electron density map particularly easily for radiotherapy planning. During radiotherapy, the intensity of the interaction of the radiation correlates with the electron density in the body, so the attenuation of the radiation as it passes through the body can be calculated relatively easily from the CT image data. This attribute is why the use of CT image data has been conventionally preferred for radiotherapy planning.

There is, however, a need for other imaging methods to be used for radiotherapy planning, such as imaging methods having a better soft part contrast, in order to allow clearer identification of target organs and/or organs at risk. One such imaging method that meets the need for better soft part contrast is magnetic resonance imaging (MR imaging) using a magnetic resonance apparatus. With this imaging modality, the contrast is a function of the distribution of the spin density, the interaction of the spins with one another and/or with their surroundings. This allows a soft part contrast to be achieved that is significantly superior to the contrast that can be achieved using computed tomography.

In a magnetic resonance apparatus, also referred to as a magnetic resonance tomography system, the body of a person to be examined, in particular a patient, is exposed to a strong basic magnetic field, for example of 1.5 or 3 or 7 Tesla, with the use of a basic field magnet. A gradient coil arrangement is also used to produce gradient pulses. Suitable antennas of a radio-frequency antenna arrangement are then operated to emit radio-frequency pulses, in particular excitation pulses, which cause the nuclear spins of certain atoms that are resonantly excited by the radio-frequency pulses to be tilted through a defined flip angle in relation to the magnetic field lines of the basic magnetic field. When the nuclear spins relax, radio-frequency signals, referred to as magnetic resonance signals, are emitted by the nuclei, and are received by the radio-frequency antenna arrangement, and then further processed. The desired image data are reconstructed from the raw data thus acquired.

A combined approach using CT imaging and magnetic resonance imaging for radiotherapy planning is known. The acquired CT image data and magnetic resonance image data are typically superimposed by an image registration procedure for radiotherapy planning. The primary benefit of CT image data in radiotherapy planning is typically the provision of electron densities and geometric precision, while magnetic resonance image data typically provide better clinical information about target organs and/or organs at risk.

In one development in recent years, referred to as MR-only RT Planning or MRORTP, the CT image data are eliminated from the planning process for suitable clinical applications. Radiotherapy planning then takes place solely based on magnetic resonance image data acquired for the patient. This allows the number of patient recordings (scans) required (only magnetic resonance scans instead of CT scans and magnetic resonance scans) to be reduced and/or possible registration errors between the CT image data and magnetic resonance image data to be avoided.

MR-only RT planning, however, presents new challenges. The determination of an electron density map for dose calculation, as required for radiotherapy planning, from conventional magnetic resonance image data requires greater algorithmic outlay than from CT image data. In contrast to CT image data, image contrasts in conventional magnetic resonance image data typically have no unique physical relationship to electron density, and therefore to photon attenuation. For example, both bone and air regions show no signal in standard magnetic resonance contrasts. Both bone regions and air regions are therefore typically both black in conventional magnetic resonance image data, even though they have different electron densities and therefore different photon attenuations.

### SUMMARY OF THE INVENTION

An object of the invention is to allow improved radiotherapy planning for a patient based on magnetic resonance measurement data.

This object is achieved by the inventive method for planning radiotherapy for a patient, which has the following method steps.

Quantitative magnetic resonance measurement data of a planning volume in the patient are acquired using a quantitative magnetic resonance.

A three-dimensional distribution of values of an electron density parameter in the planning volume is determined in a processor based on the acquired quantitative magnetic resonance measurement data.

A radiotherapy plan is calculated in the processor using the three-dimensional distribution of the values of the electron density parameter, and the calculated plan is made available from the computer as an electronic output signal.

The acquisition of the quantitative magnetic resonance measurement data of the planning volume can be the scanning of quantitative magnetic resonance measurement data using a magnetic resonance scanner, or downloading already-recorded quantitative magnetic resonance measurement data from a database into the processor. The calculated radiotherapy plan can be provided for implementing the radiotherapy on the patient. To this end, the calculated radiotherapy plan can be transmitted to a radiotherapy application, for example a linear accelerator or a brachytherapy source, so the radiotherapy can be performed. The calculated radiotherapy plan can also be stored in a database for subsequent retrieval.

The planning volume encompasses the target volume for the radiotherapy and at least one volume at risk for the radiotherapy. The target volume and/or the at least one volume at risk can be marked semi-automatically or manually in the magnetic resonance image data acquired for the planning volume and/or in the first electron density map, for example by plotting a point or a 2D or 3D region of interest (ROI). The planning volume is therefore typically selected such that it is large enough for the target volume and the at least one volume at risk to be included in the planning volume under any circumstance. If, for example, the patient's prostate is selected for radiotherapy, the planning volume can include the patient's entire pelvic region. The target volume includes the target structures in the patient's body to be irradiated by the beam therapy. The target tissue is typically located in the target volume. A beam dose can be allocated to the target volume during radiotherapy planning. The target volume is typically delineated by the at least one volume at risk. The at least one volume at risk involves tissue at risk for the radiotherapy. A maximum dose can be allocated to the at least one volume at risk during radiotherapy planning, and it is not permitted for this maximum dose to be exceeded during the beam therapy.

The quantitative magnetic resonance method used to acquire the magnetic resonance image data serves to determine at least one quantitative tissue parameter. The at least one tissue parameter is quantified with local spatial resolution. This means that a spatially locally resolved distribution of the at least one tissue parameter is quantified. The at least one tissue parameter preferably characterizes a physical attribute of the matter, for example the tissue, for which the magnetic resonance signals are acquired. The at least one tissue parameter can quantify a reaction of the matter to a radio-frequency excitation. Possible tissue parameters that can be quantified using the quantitative magnetic resonance method are an electron density (for example a linear attenuation coefficient), a T1 relaxation time, a T2 relaxation time, a diffusion value (for example an apparent diffusion coefficient, ADC), a magnetization moment, a proton density, a resonant frequency, a concentration of particular (selected) matter, etc. Further tissue parameters that appear expedient to those skilled in the art are also conceivable. Any combination (selection) of the cited tissue parameters can be determined using the quantitative magnetic resonance method.

The quantitative magnetic resonance method allows quantification of the at least one tissue parameter, for example independently of measuring conditions or a type of magnetic resonance device. The quantification of the at least one tissue parameter thus can be independent of parameter settings, calibration measurements, coil intensities, software versions, etc. A quantitative magnetic resonance image of the magnetic resonance image data reconstructed from a quantitative magnetic resonance method thus can contain information about absolute physical variables. Thus a value of an image pixel of such a quantitative magnetic resonance image is directly related to a physical measurement value. The value of an image pixel can be expressed by a physical unit. It is thus possible to compare magnetic resonance images recorded using different quantitative magnetic resonance methods, possibly under different measuring conditions, directly with one another.

The three-dimensional distribution of the values of the electron density parameter is preferably a locally spatially resolved distribution of electron density. The three-dimensional distribution of the values of the electron density parameter can also be referred to as an electron density map. The inputs in the three-dimensional distribution of the values of the electron density parameter can be in units that, for example, characterize electron density, for example a linear attenuation coefficient with a unit of 1/m or Hounsfield units. The electron density parameter thus can be formed by a linear attenuation coefficient and be measured in Hounsfield units. The three-dimensional distribution of the values of the electron density parameter can first describe the electron density resulting from the tissue of the patient as located in the planning volume. A method known to those skilled in the art can then be used to integrate information about the distribution of additional values of the electron density parameter in the planning volume due to hardware components of the radiotherapy apparatus, for example a patient support apparatus, in the three-dimensional distribution of the values of the electron density parameter.

To determine the three-dimensional distribution of the values of the electron density parameter it is possible to use a determination algorithm that accepts input parameters in the form of the quantitative magnetic resonance measurement data, and that produces output parameters in the form of the three-dimensional distribution of the values of the electron density parameter. The determination algorithm can allocate values of the electron density parameter to voxels of the quantitative magnetic resonance measurement data here based on the signal values determined using the quantitative magnetic resonance method for said voxel. Different exemplary options for determining the three-dimensional distribution of the values of the electron density parameter from the quantitative magnetic resonance measurement data are described below.

The three-dimensional distribution of the values of the electron density parameter can also be referred to as a virtual or synthetic computed tomography image (CT image). The determination of the three-dimensional distribution of the values of the electron density parameter can thus comprise the production of the virtual CT image from the quantitative magnetic resonance measurement data. The virtual CT image is generated solely using the quantitative magnetic resonance measurement data. The virtual CT image can represent medical image data acquired in the same patient geometry of the quantitative magnetic resonance measurement data. However the virtual CT image can also have a gray-scale value distribution that would be present if CT image data of the same patient had been acquired. The virtual CT image or the three-dimensional distribution of the values of the electron density parameter can then be used for MR-only RT planning. The CT measurement or x-ray measurement actually required for radiotherapy planning thus can be simulated using the quantitative magnetic resonance measurement data. The calculation of the radiotherapy plan using the three-dimensional distribution of the values of the electron density parameter then can be performed using a method known to those skilled in the art, automatically or semi-automatically.

The three-dimensional distribution of the values of the electron density parameter in the planning volume thus can be determined solely on the basis of the quantitative magnetic resonance measurement data. No further medical measurement data, such as from a different imaging modality, are used, apart from the quantitative magnetic resonance measurement data, to determine the three-dimensional distribution of the values of the electron density parameter. There is thus no need for computed tomography measurements when planning radiotherapy for the patient. This reduces the time and cost when planning radiotherapy for the patient and there is no need for an additional dose of radiation for the patient. The work flow can also be simplified, as it is only necessary to record measurement data from a single modality, specifically quantitative magnetic resonance measurement data, for radiotherapy planning. Radiotherapy planning errors due to the patient being supported differently during magnetic resonance measurements and CT measurements thus can be avoided.

The virtual CT image or the three-dimensional distribution of the values of the electron density parameter can also be used for improved automatic registration of CT image data and magnetic resonance image data for radiotherapy planning. To this end, the virtual CT image can be aligned with a CT image actually acquired for the patient. Resulting transformation formulae can be used to transform the magnetic resonance image data for radiotherapy planning.

In an embodiment the acquisition of the quantitative magnetic resonance measurement data includes a quantification of a measurement n-tuple of tissue parameters for at least one voxel in the planning volume. In this embodiment the determination of the three-dimensional distribution of the values of the electron density parameter is a value comparison of the measurement n-tuple with tissue n-tuples for a number of tissue types stored in a tissue database and an assignment of one of the number of tissue types to the at least one voxel based on a result of the value comparison.

In one typical application, this described procedure is performed for all voxels of the planning volume. The tissue parameters are quantified here using the magnetic resonance signals acquired with the quantitative magnetic resonance method. The quantification of the tissue parameters is performed with local spatial resolution. The quantified tissue parameters are then brought together in the measurement n-tuple. Thus a first input in the measurement n-tuple can be a first quantified tissue parameter, a second input in the measurement n-tuple can be a second quantified tissue parameter, etc. It is also conceivable for just one tissue parameter to be quantified, so that a measurement 1-tuple is quantified. It is, however, advantageous for a number of tissue parameters to be quantified. The number of tissue parameters in the measurement n-tuple is therefore preferably greater than 1.

The tissue n-tuples stored in the tissue database can be the tissue parameters of the number of tissue types. Tissue types here can be typical body tissue occurring in a human body, for example gray brain matter, white brain matter, fatty tissue, soft part tissue, bone tissue, lung tissue, etc. A tissue n-tuple is stored in the tissue database for each tissue type. The tissue n-tuples can be determined in advance by measurement or based on a priori knowledge of the material attributes of the tissue types. The tissue parameter values stored in the tissue n-tuples can be all of the physical attributes of the respective tissue types, as required for the determination of the appropriate tissue type based on the measurement n-tuple. The value comparison of the measurement n-tuple can then be performed with all the tissue n-tuples of the different tissue types. The value comparison can be a comparison of the measurement values of the tissue parameter stored in the measurement n-tuple with the tissue values of the tissue parameter stored in the tissue n-tuples. The tissue type of the number of tissue types, the tissue n-tuple of which corresponds best in the value comparison to the measurement n-tuple, can then be assigned to the at least one voxel.

A distribution of the tissue types in the planning volume or a planning volume segmented into the number of tissue types can be determined as a result of this procedure. It is then particularly simple to determine the appropriate values of the electron density parameter for the voxels of the planning volume based on the tissue types assigned to the voxels of the planning volume, as described in more detail below. It is thus particularly simple to determine the three-dimensional distribution of the values of the electron density parameter in the planning volume based on the acquired quantitative magnetic resonance measurement data.

In an embodiment, the acquisition of the quantitative magnetic resonance measurement data is implemented by the use of a magnetic resonance fingerprinting method, in order to quantify the measurement n-tuple.

One possible magnetic resonance fingerprinting method is known, for example, from the publication Ma et al., “Magnetic Resonance Fingerprinting”, Nature, 495, 187-192 (14 Mar. 2013). With a magnetic resonance fingerprinting method, recording parameters are typically varied in a pseudo-randomized manner during the acquisition of the quantitative magnetic resonance measurement data. Possible recording parameters that are changed during the acquisition of the quantitative magnetic resonance measurement data are, for example, an echo time, a formation and/or number of radio-frequency pulses, a formation and/or number of gradient pulses, a diffusion coding, etc. A magnetic resonance signal profile is then typically generated for the at least one voxel over the duration of the acquisition of the quantitative magnetic resonance measurement data. The magnetic resonance signal profile therefore indicates a change in recorded magnetic resonance signal values over the duration of the acquisition of the magnetic resonance signal profile.

The magnetic resonance signal profile acquired for the at least one voxel is then compared, for the quantification of the measurement n-tuple, with a number of database signal profiles stored in a magnetic resonance fingerprinting database in a signal comparison. The abovementioned tissue database here can be a different database from the magnetic resonance fingerprinting database. A different database value of at least one tissue parameter is assigned here to each of the different database signal profiles. The database signal profile then represents the signal profile expected in each instance with the magnetic resonance fingerprinting method, when a sample, the material attributes of which correspond to those of the associated database value of the at least one tissue parameter, is examined. The database signal profiles can be determined and/or simulated, for example in a calibration measurement. In the magnetic resonance fingerprinting method there is then typically provision for one of the number of database signal profiles to be assigned to the generated magnetic resonance signal profile based on the result of the signal comparison.

The database value of the at least one tissue parameter belonging to the assigned database signal profile can then be set as the measurement value of the at least one tissue parameter. The magnetic resonance fingerprinting method advantageously allows the simultaneous quantification of a number of tissue parameters for the at least one voxel. This allows the measurement n-tuple to be determined particularly precisely and/or quickly for the at least one voxel using the magnetic resonance fingerprinting method.

In another embodiment the acquisition of the quantitative magnetic resonance measurement data, acquisition of a magnetic resonance signal profile of at least one voxel in the planning volume is implemented using a magnetic resonance fingerprinting method, and the determination of the three-dimensional distribution of the values of the electron density parameter is implemented by a signal comparison of the magnetic resonance signal profile with tissue signal profiles of a number of tissue types stored in a tissue database and an assignment of one of the number of tissue types to the at least one voxel based on a result of the signal comparison.

In a typical application, the described procedure is performed for all the voxels of the planning volume. The fundamental mode of operation of the magnetic resonance fingerprinting method, in particular the acquisition of the magnetic resonance signal profile, is described below. In accordance with the invention, however, the quantification of the measurement n-tuple of the tissue parameters using the magnetic resonance fingerprinting method can be omitted. Instead, the present tissue type can be determined directly by the signal comparison of the magnetic resonance signal profile with the tissue signal profiles for the at least one voxel. There is thus no need for an alternative approach by the quantification of the measurement n-tuple using the magnetic resonance fingerprinting method and the comparison of the measurement n-tuple with the tissue n-tuples.

In accordance with the invention, the tissue database is therefore configured differently from in the application described above. The appropriate tissue signal profiles for the respective tissue types are stored in the tissue database here and not the tissue n-tuples. It is also conceivable to combine both applications to improve accuracy and to store both appropriate tissue signal profiles and appropriate tissue n-tuples for the respective tissue types.

It is also possible to determine a distribution of the tissue types in the planning volume or a planning volume segmented into the number of tissue types using this procedure. The tissue types assigned to the voxels of the planning volume can then be used to determine the appropriate values of the electron density parameter for the voxels of the planning volume in a particularly simple manner, as described below. It is thus particularly easy to determine the three-dimensional distribution of the values of the electron density parameter in the planning volume based on the acquired quantitative magnetic resonance measurement data.

In another embodiment, different values of the electron density parameter are stored in the tissue database for the number of tissue types, with the value of the electron density parameter stored for the tissue type assigned to the at least one voxel being set for the at least one voxel in the three-dimensional distribution of the values of the electron density parameter.

In a typical application, the described procedure is performed for all the voxels of the planning volume. The starting point is therefore in particular a distribution of the tissue types in the planning volume or a planning volume segmented into the number of tissue types. This can be determined from the quantitative magnetic resonance measurement data using one of the described procedures.

The respective value of the electron density parameter of the tissue type is also stored in the tissue database in particular for each tissue type. As described in the following paragraph, a number of values of the electron density parameter can also be stored for the tissue type. The values of the electron density parameter stored for each of the tissue types can be used particularly easily to draw conclusions about the three-dimensional distribution of the values of the electron density parameter in the planning volume from the planning volume segmented into the number of tissue types.

In an embodiment, at least two different values of the electron density parameter, to which different irradiation energies used for radiotherapy for the patient are assigned, are stored in the tissue database for at least one of the number of tissue types.

This procedure is based on the knowledge that the tissue of the patient typically absorbs radiation to differing degrees at different irradiation energies. A first value of the electron density parameter can thus be stored for a first irradiation energy and a second value of the electron density parameter can be stored for a second irradiation energy. Exemplary irradiation energies here are: 120 kV, 6 MeV, 15 MeV. Other irradiation energies are also conceivable. The value of the electron density parameter assigned to the irradiation energy used for radiotherapy for the patient is then used for the determination of the three-dimensional distribution of the values of the electron density parameter. This allows the calculated distribution of the values of the electron density parameter to be tailored in a particularly suitable manner to basic conditions implemented during the patient's radiotherapy.

Similarly, it is also conceivable for at least two different values of the electron density parameter, to which different radiotherapy modalities used for the patient's radiotherapy are assigned, to be stored in the tissue database for at least one of the number of tissue types. Possible radiotherapy modalities are, for example, radiotherapy with photons, radiotherapy with electrons, radiotherapy with protons, radiotherapy with ions, etc. The appropriate value of the electron density parameter can then be used for radiotherapy planning based on the radiotherapy modality used.

In another embodiment, after the patient has been positioned on a patient support apparatus of a radiotherapy apparatus, which is used for the patient's radiotherapy, the position of the patient is monitored by comparing the three-dimensional distribution of the values of the electron density parameter with control image data, with the control image data being recorded using an imaging unit of the radiotherapy apparatus.

The position of the patient is monitored before the irradiation of the patient by the radiotherapy apparatus. The imaging device of the radiotherapy apparatus can be, for example, a cone beam computed tomography emitter or an x-ray radiator with kilovolt or megavolt radiation energies. To acquire the control image data, the imaging device can be arranged in direct spatial proximity to the radiotherapy apparatus or integrated in the radiotherapy apparatus. The control image data used can be the image data generally used for a manual position check. Additional control image data can also be recorded using the imaging device of the radiotherapy apparatus specifically for the described position check.

The comparison of the three-dimensional distribution of the values of the electron density parameter with the control image data can be a superimposed or merged representation of the three-dimensional distribution of the values of the electron density parameter with the control image data. The comparison can then be performed by a competent person inspecting the superimposed or merged representation. The similar image impression of the three-dimensional distribution of the values of the electron density parameter and the control image data can simplify the inspection. Computer-based automatic or semi-automatic comparisons of the three-dimensional distribution of the values of the electron density parameter with the control image data are also conceivable.

In an embodiment, the determination of a three-dimensional distribution of values of an electron density parameter comprises a use of basic conditions, which determine limits for a value of the of the electron density parameter for at least one voxel of the planning volumes.

Such a procedure can facilitate the assignment of the values of the electron density parameters to the information determined from the quantitative magnetic resonance method. It is thus possible to avoid or eliminate non-unique assignments between the quantitative magnetic resonance measurement data and the values of the electron density parameter. For example the anatomical position of at least one voxel in the quantitative magnetic resonance measurement data in the body of the patient can be used to determine the basic conditions for the value of the electron density parameter. It is thus possible, for example, to exclude value ranges determined in this manner for the value of the electron density parameter. An atlas-based procedure can also be used to determine the anatomical position of the at least one voxel in the body of the patient, such as registration of an atlas with the quantitative magnetic resonance measurement data.

The inventive radiotherapy planning computer has an acquisition processor, a determination processor and a calculation processor. The radiotherapy planning computer is thus configured to execute an inventive method.

The radiotherapy planning computer is thus configured to execute the above-described method for planning radiotherapy for a patient. The acquisition processor is configured to receive quantitative magnetic resonance measurement data of a planning volume in the patient using a quantitative magnetic resonance method. The determination processor is configured to determine a three-dimensional distribution of values of an electron density parameter in the planning volume based on the acquired quantitative magnetic resonance measurement data. The calculation processor is configured to calculate a radiotherapy plan using the three-dimensional distribution of the values of the electron density parameter.

The components of the radiotherapy planning computer, specifically the acquisition processor, determination processor and calculation processor, can largely be configured in the form of software components. However in principle some of these components, such as when particularly fast calculations are required, can be implemented in the form of software-supported hardware components, for example FPGAs or the like. Similarly the required interfaces can be configured as software interfaces, for example when it is simply a matter of copying data over from other software components. However, the interfaces also can be configured as hardware-based interfaces, which are activated by appropriate software. It is also conceivable for a number of the cited components to be implemented in a combined manner in the form of an individual software component or software-supported hardware component.

The inventive magnetic resonance apparatus has an inventive radiotherapy planning computer. The radiotherapy planning computer can be configured to send control signals to the magnetic resonance scanner and/or to receive and/or process control signals, in order to execute the inventive method. The radiotherapy planning computer can be integrated into the control computer of the magnetic resonance apparatus. The radiotherapy planning computer can also be installed separately from the magnetic resonance apparatus. The radiotherapy planning computer can be connected to the magnetic resonance apparatus. Acquisition of the quantitative magnetic resonance measurement data can be the recording of the quantitative magnetic resonance measurement data using the scanner of the magnetic resonance apparatus. The quantitative magnetic resonance measurement data can then be forwarded to the radiotherapy planning computer for further processing. The radiotherapy planning computer then receives the quantitative magnetic resonance measurement data via the acquisition processor.

The invention also encompasses a non-transitory, computer-readable data storage medium encoded with programming instructions that, when the storage medium is loaded into a radiotherapy planning computer, or a control computer of a magnetic resonance apparatus, cause the radiotherapy planning computer and/or the control computer to implement the method as described above, when the programming instructions are executed.

Examples of electronically readable data media are a DVD, a magnetic tape or a USB stick, on which electronically readable control information, in particular software is stored.

The advantages of the inventive radiotherapy planning computer, the inventive magnetic resonance apparatus and the inventive data storage medium correspond essentially to the advantages of the inventive method, described in detail above. Features, advantages or alternative embodiments mentioned in this context area applicable to the other aspects of the invention. The corresponding functional features of the method are formed here by corresponding object-based modules, in particular by hardware modules.

### BRIEF DESCRIPTION OF THE DRAWINGS

FIG. 1 shows an inventive magnetic resonance device having an inventive radiotherapy planning computer.

FIG. 2 shows a first embodiment of an inventive method.

FIG. 3 shows a second embodiment of an inventive method.

### DESCRIPTION OF THE PREFERRED EMBODIMENTS

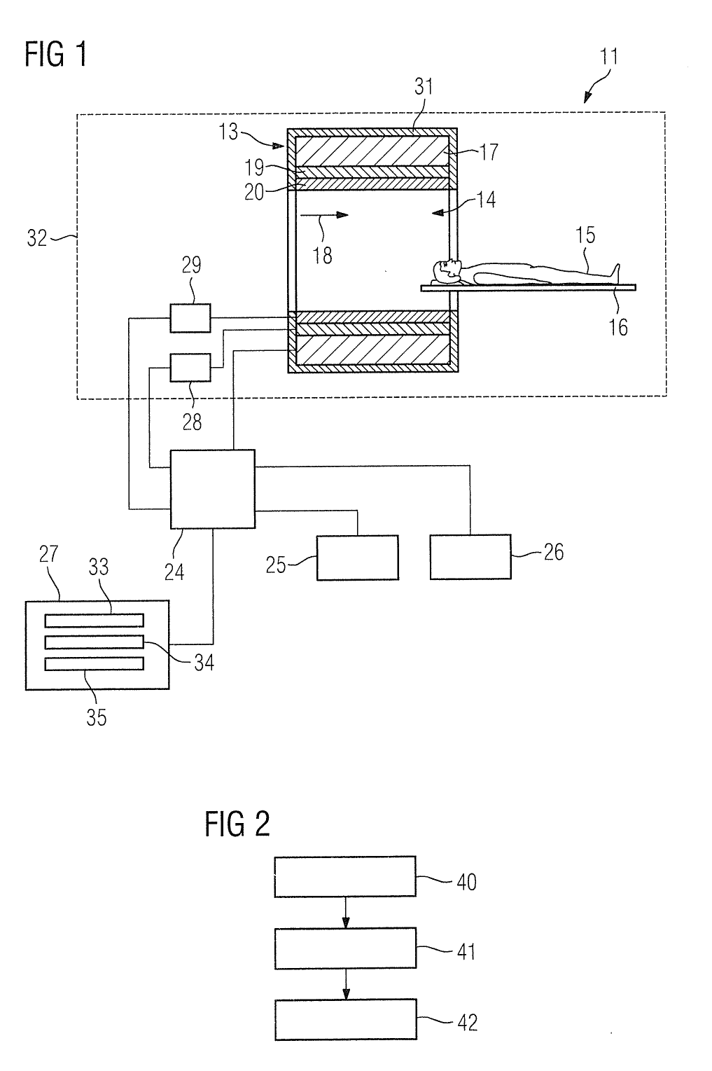

FIG. 1 is a schematic illustration of an inventive magnetic resonance apparatus 11. The magnetic resonance apparatus 11 has a data acquisition scanner formed by a magnet unit 13 with a basic field magnet 17 for generating a powerful and constant basic magnetic field 18. The magnetic resonance scanner has a cylindrical patient receiving region 14 for receiving a patient 15, the patient receiving region 14 being enclosed cylindrically in a peripheral direction by the magnet unit 13. The patient 15 can be moved into the patient receiving region 14 by a patient support 16. The patient support apparatus 16 has a couch, which is movable within the patient receiving region 14. The magnet unit 13 is shielded externally by a housing shell 31.

The magnet unit 13 also has a gradient coil arrangement 19 for generating magnetic field gradients, which are used for spatial encoding during imaging. The gradient coil arrangement 19 is activated by a gradient control processor 28. The magnet unit 13 also has a radio-frequency antenna 20, which is configured as a body coil integrated in a fixed manner in the scanner in the illustrated instance, and a radio-frequency antenna control processor 29 for exciting certain nuclear spins so as to deviate from the polarization established in the basic magnetic field 18 generated by the basic field magnet 17. The radio-frequency antenna 20 is activated by the radio-frequency antenna control processor 29 and to radiate radio-frequency magnetic resonance sequences into an examination chamber formed essentially by the patient receiving region 14. The radio-frequency antenna 20 is also configured to receive magnetic resonance signals from the patient 15 that result as the excited nuclear spins relax.

The magnetic resonance apparatus 11 has a control computer 24 for controlling the basic field magnet 17, the gradient control processor 28 and the radio-frequency antenna control processor 29. The control computer 24 controls the magnetic resonance apparatus 11 centrally, for example in the performance of a predefined imaging gradient echo sequence. Control information such as imaging parameters for example, as well as reconstructed magnetic resonance images, can be provided for a user on an input interface 25, in the present instance a display monitor, of the magnetic resonance apparatus 11. The magnetic resonance apparatus 11 also has an input interface 26, via which a user can enter information and/or parameters during a measuring procedure. The control computer 24 can comprise the gradient control processor 28 and/or radio-frequency antenna control processor 29 and/or the output interface 25 and/or the input interface 26.

The illustrated magnetic resonance apparatus 11 can have further components that are generally used in magnetic resonance apparatuses. A general mode of operation of a magnetic resonance apparatus 11 is also known to the person skilled in the art, so there is no need to describe the further components in detail here. The magnetic resonance apparatus 11 can also be configured as a combined magnetic resonance device and linear accelerator (MR-LINAC) device. This means that the radiotherapy plan produced by the magnetic resonance apparatus 11 can then be used for radiotherapy for the patient 15 in the same device.

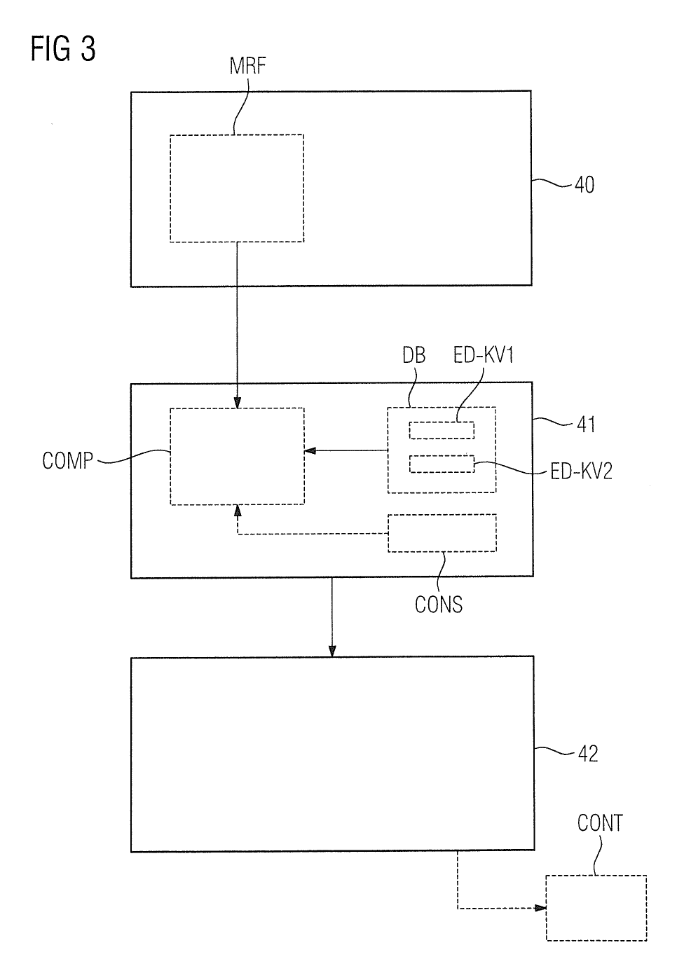

The illustrated magnetic resonance apparatus 11 has a radiotherapy planning computer 27, which has an acquisition processor 33, a determination processor 34 and a calculation processor 35. The radiotherapy planning computer 27 is configured to execute the method according to FIGS. 2-3.

For the sole execution of an inventive method the radiotherapy planning computer 27 will download quantitative magnetic resonance measurement data from a database via the acquisition processor 33. When the inventive method is executed in a combined manner by the magnetic resonance apparatus 11 with the radiotherapy planning computer 27, the acquisition processor 33 of the radiotherapy planning computer 27 will receive quantitative magnetic resonance measurement data recorded by the scanner of the magnetic resonance apparatus 11. To this end, the radiotherapy planning computer 27, in particular the acquisition processor 33, is connected for data exchange to the control computer 24 of the magnetic resonance apparatus 11.

FIG. 2 is a flowchart of a first embodiment of an inventive method for planning radiotherapy for a patient 15.

In a first method step 40 quantitative magnetic resonance measurement data of a planning volume in the patient 15, acquired using a quantitative magnetic resonance method, are received by the acquisition processor 33.

In a further method step 41, a three-dimensional distribution of values of an electron density parameter in the planning volume is determined based on the acquired quantitative magnetic resonance measurement data, by the determination processor 34.

In a further method step 42, a radiotherapy plan is calculated using the three-dimensional distribution of the values of the electron density parameter, by the calculation processor 35.

FIG. 3 shows a flowchart of a second embodiment of an inventive method for planning radiotherapy for a patient 15.

The description which follows is essentially limited to the differences compared with the exemplary embodiment in FIG. 2, reference being made to the description of the exemplary embodiment in FIG. 2 for method steps that remain the same. Method steps that remain essentially the same are in principle shown with the same reference characters.

The embodiment of the inventive method shown in FIG. 3 essentially includes the method steps 40, 41, 42 of the first embodiment of the inventive method according to FIG. 2. The embodiment of the inventive method shown in FIG. 3 also comprises additional method steps and sub-steps. An alternative method sequence to the one in FIG. 3, which only has some of the additional method steps and/or sub-steps shown in FIG. 3, is also conceivable. Of course an alternative method sequence to the one in FIG. 3 can also have additional method steps and/or sub-steps.

In the first method step 40, the acquisition of the quantitative magnetic resonance measurement data in the embodiment illustrated in FIG. 3 is implemented by the use of a magnetic resonance fingerprinting method MRF.

In a first possible embodiment the acquisition of the quantitative magnetic resonance measurement data can be a quantification of a measurement n-tuple of tissue parameters for at least one voxel in the planning volume using the magnetic resonance fingerprinting method MRF.

In the further method step 41, the determination of the three-dimensional distribution of the values of the electron density parameter will then be a value comparison COMP of the measurement n-tuple with tissue-n-tuples of a number of tissue types stored in a tissue database DB, and an assignment of one of the number of tissue types to the at least one voxel based on a result of the value comparison COMP.

In a second embodiment the acquisition of the quantitative magnetic resonance measurement data in the first method step 40 is the acquisition of a magnetic resonance signal profile of at least one voxel in the planning volume using the magnetic resonance fingerprinting method MRF.

The determination of the three-dimensional distribution of the values of the electron density parameter in the further method step 41 will then be a signal comparison COMP of the magnetic resonance signal profile with tissue signal profiles of a number of tissue types stored in a tissue database DB and an assignment of one of the number of tissue types to the at least one voxel based on a result of the signal comparison COMP.

As can be seen, the first application and the second application differ with respect to the initial data of the magnetic resonance fingerprinting method MRF. In the first application this is a measurement n-tuple of tissue parameters, while in the second application the initial data is formed by a magnetic resonance signal profile. The first application and the second application also differ with respect to the comparison COMP and the inputs in the tissue database DB in the further method step 41. In the first application a value comparison COMP is performed between the measurement n-tuple and tissue-n-tuples stored in the tissue database DB. In the second application the signal comparison COMP of the magnetic resonance signal profile is performed with tissue signal profiles stored in the tissue database DB. However in both embodiments one of the number of tissue types is assigned to the voxel in the further method step 41. The first application and the second application here can be deployed separately from one another, in other words one procedure can be selected from the two embodiments. It is also conceivable for the first application and the second application to be deployed together in a combined manner.

Different values of the electron density parameter are stored in the tissue database DB for the number of tissue types, with the value of the electron density parameter stored for the tissue type assigned to the at least one voxel being set in the further method step 41 for the at least one voxel in the three-dimensional distribution of the values of the electron density parameter. This procedure is equally possible for both embodiments. At the same time at least two different values of the electron density parameter, to which different irradiation energies used for radiotherapy for the patient 15 are assigned, can be stored in the tissue database DB for at least one of the number of tissue types. A first set of values of the electron density parameter ED-KV1, which is assigned to a first irradiation energy, for example 6 MeV, is therefore conceivable, as is a second set of values of the electron density parameter ED-KV2, which is assigned to a second irradiation energy, for example 15 MeV. Based on the irradiation energy used for radiotherapy for the patient 15, the appropriate set of values of the electron density parameter ED-KV1, ED-KV2 can be used to determine the three-dimensional distribution of the values of the electron density parameter. At the same time in certain applications the determination of the three-dimensional distribution of the values of the electron density parameter in the further method step 41 can comprise the use of basic conditions CONS, which determine limits for the value of the electron density parameter for the at least one voxel of the planning volume.

Optionally, in a further method step CONT after the patient 15 has been positioned on a patient support apparatus of a radiotherapy apparatus used for radiotherapy for the patient 15, the position of the patient can be monitored by a comparison of the three-dimensional distribution of the values of the electron density parameter with control image data, with the control image data being recorded using an imaging device of the radiotherapy apparatus.

The method steps of the inventive method illustrated in FIGS. 2-3 are executed by the radiotherapy planning computer 27. To this end the radiotherapy planning computer 27 has the necessary software and/or computer programs, which are stored in a memory of the radiotherapy planning computer 27. The software and/or computer programs include program code designed to cause the inventive method to be implemented when the computer code is executed in the radiotherapy planning computer 27.

Although modifications and changes may be suggested by those skilled in the art, it is the intention of the Applicant to embody within the patent warranted hereon all changes and modifications as reasonably and properly come within the scope of the Applicant's contribution to the art.

1. A method for planning radiotherapy for a patient, comprising:
providing a processor with quantitative magnetic resonance measurement data of a planning volume in a patient, which were acquired using a quantitative magnetic resonance data acquisition method;
in said processor, automatically determining a three-dimensional distribution of values of an electron density parameter in said planning volume based on the acquired quantitative magnetic resonance measurement data;
in said processor, automatically calculating a radiotherapy plan using the three-dimensional distribution of the values of said electron density parameter; and
emitting an electronic signal from said processor that represents said radiotherapy plan.

2. A method as claimed in claim 1 comprising:
providing said processor with a quantification of a measurement n-tuple of tissue parameters for at least one voxel in said planning volume, as said quantitative magnetic resonance measurement data; and
in said processor, determining said three-dimensional distribution of the values of said electron density parameter by making a value comparison of the measurement n-tuple with tissue n-tuples for a plurality of tissue types stored in a tissue database accessible by said processor, and by making an assignment of one of said plurality of tissue types to said at least one voxel based on a result of said value comparison.

3. A method as claimed in claim 2 comprising providing said quantitative magnetic resonance measurement data to said processor as data acquired by a magnetic resonance fingerprinting method, and using said data from said magnetic resonance fingerprinting method to quantify to said measurement n-tufle.

4. A method as claimed in claim 2 comprising storing different values of said electron density parameter in said tissue database respectively for said plurality of tissue types, with the value of the electron density parameter stored for a respective tissue type assigned to said at least one voxel being set for said at least one voxel in said three-dimensional distribution of the values of said electron density parameter.

5. A method as claimed in claim 4 comprising storing at least two different values of said electron density parameter in said tissue database for at least one of said plurality of tissue types, and assigning different irradiation energies for said radiotherapy respectively to said at least two different values of said electron density parameter.

6. A method as claimed in claim 1 comprising:
providing said processor with said quantitative magnetic resonance measurement data as magnetic resonance fingerprinting data and, in said processor, acquiring a magnetic resonance signal profile of at least one voxel in said planning volume using said magnetic resonance fingerprinting data; and
in said processor, determining said three-dimensional distribution of the values of said electron density parameter by performing a signal comparison of the magnetic resonance signal profile with tissue-signal profiles for a plurality of tissue types stored in a tissue database, accessible by said processor, and assigning one of said plurality of tissue types to said at least one voxel based on a result of said signal comparison.

7. A method as claimed in claim 6 comprising storing different values of said electron density parameter in said tissue database respectively for said plurality of tissue types, with the value of the electron density parameter stored for a respective tissue type assigned to said at least one voxel being set for said at least one voxel in said three-dimensional distribution of the values of said electron density parameter.

8. A method as claimed in claim 7 comprising storing at least two different values of said electron density parameter in said tissue database for at least one of said plurality of tissue types, and assigning different irradiation energies for said radiotherapy respectively to said at least two different values of said electron density parameter.

9. A method as claimed in claim 1 comprising, after positioning the patient on a patient support apparatus of a radiotherapy apparatus that is used to implement said radiotherapy on said patient, monitoring the position of the patient by comparing the three-dimensional distribution of the values of said electron density parameter with control image data provided to said processor, said control image data being recorded using an imaging device of said radiotherapy apparatus.

10. A method as claimed in claim 1 comprising determining said three-dimensional distribution of values of said electron density parameter using basic conditions that determine limits for said value of said electron density parameter for at least one voxel of said planning volume.

11. A radiotherapy planning computer comprising:
an input interface configured to receive quantitative magnetic resonance measurement data of a planning volume in a patient, which were acquired using a quantitative magnetic resonance data acquisition method;
a processor in communication with said input interface, said processor being configured to automatically determine a three-dimensional distribution of values of an electron density parameter in said planning volume based on the acquired quantitative magnetic resonance measurement data;
said processor being configured to automatically calculate a radiotherapy plan using the three-dimensional distribution of the values of said electron density parameter; and
an output interface in communication with said processor, configured to emit an electronic signal that represents said radiotherapy plan.

12. A magnetic resonance apparatus comprising:
a magnetic resonance data acquisition scanner;
a control computer configured to operate the data acquisition scanner to acquire quantitative magnetic resonance measurement data of a planning volume in a patient using a quantitative magnetic resonance data acquisition method;
said control computer being configured to automatically determine a three-dimensional distribution of values of an electron density parameter in said planning volume based on the acquired quantitative magnetic resonance measurement data;
said control computer being configured to automatically calculate a radiotherapy plan using the three-dimensional distribution of the values of said electron density parameter; and
said control computer being configured to emit an electronic signal from said processor that represents said radiotherapy plan.

13. A non-transitory, computer-readable data storage medium encoded with programming instructions, said storage medium being loaded into a control computer of a magnetic resonance apparatus, said programming instructions causing said control computer to:
receive quantitative magnetic resonance measurement data of a planning volume in a patient, which were acquired using a quantitative magnetic resonance data acquisition method;
automatically determine a three-dimensional distribution of values of an electron density parameter in said planning volume based on the acquired quantitative magnetic resonance measurement data;
automatically calculate a radiotherapy plan using the three-dimensional distribution of the values of said electron density parameter; and
emit an electronic signal from said processor that represents said radiotherapy plan.
